(19) United States
(12) Patent Application Publication
(10) Pub. No.: US 2022/0375326 A1
(43) Pub. Date: Nov. 24, 2022

(54) Device For Reducing Face-Touching

(71) Applicant: Brian Basham, Denver, CO (US)

(72) Inventor: Brian Basham, Denver, CO (US)

(21) Appl. No.: 17/750,286

(22) Filed: May 20, 2022

Related U.S. Application Data
(60) Provisional application No. 63/191,241, filed on May 20, 2021.

(51) Int. Cl. G08B 21/24; G08B 5/38; G08B 6/00; G08B 7/06; G16H 40/20

(52) U.S. Cl. CPC G08B 21/245; G08B 5/38; G08B 6/00; G08B 7/06; G16H 40/20

(57) ABSTRACT

A device for reducing face-touching. The device includes a battery, a sensor for detecting when a user's hand is within a threshold proximity to the user's face, a vibrator configured to vibrate in response to the detection by the sensor that the user's hand is within the threshold proximity, and a clip configured to attach the device to an item worn by the user. The battery, sensor, and vibrator are contained with the same casing.

### TECHNICAL FIELD

The present disclosure generally relates to a device for reducing face-touching.

### BACKGROUND

Most people subconsciously touch their faces with their hands several times a day. For example, people often rub their eyes, scratch their noses, or bite their nails. This can allow germs on the hands to enter the body through mucous membranes in the eyes, nose, and mouth. These germs have the potential to cause serious respiratory illnesses such as COVID-19.

### SUMMARY

Embodiments of the present disclosure can provide a way to train people to limit the number of times they touch their faces. This can help prevent the spread of respiratory illnesses such as the flu, the common cold, and COVID-19. In addition, this can help with obsessive-compulsive disorders such as chronic skin-picking. Further, this can help with other mental disorders such as compulsive over-eating.

For instance, the disclosure provides a small sensing unit that uses infrared and/or ultrasonic sensors to detect movement of a person's hands toward their face. The sensors may be embedded in the device with a small battery. The device generates a vibration similar to a cell phone and/or flashes a small LED light when a person's hands come close to their face. This alerts and conditions the person wearing the device not to touch their face. The device can be attached to sunglasses, a hat, a shirt collar, or a mask.

The sensing unit can be worn by hospital staff during surgeries or patient interactions as part of their daily routine. For example, hospital staff can wear the sensing unit to protect themselves from possible pathogens that patients may be carrying.

The disclosure further provides for devices for reducing face-touching. In an exemplary embodiment, a device includes a battery, a sensor for detecting when a user's hand is within a threshold proximity to the user's face, a vibrator configured to vibrate in response to the detection by the sensor that the user's hand is within the threshold proximity, and a clip configured to attach the device to an item worn by the user. The battery, sensor, and vibrator are contained with the same casing.

In the above embodiment, the device may further include an LED light configured to light up when the sensor detects that the user's hand is within the threshold proximity. The device may further include a switch for activating and deactivating the LED light. The battery may be a rechargeable battery. The vibrator may be configured to generate a vibration output with increasing intensity as the user's hand gets closer to the user's face. The device may further include a switch for controlling the sensitivity of the sensor to adjust the threshold proximity. The device may further include a switch for controlling a vibration intensity of the vibrator. The sensor may be an infrared sensor. The sensor may be an ultrasonic sensor. The device may further include a processor configured to process information obtained from the sensor and to cause the vibrator to generate a vibration output. The clip may be configured to attach the device to a collar of a shirt worn by the user. The clip may be configured to attach the device to a hat worn by the user. The clip may be configured to attach the device to a mask worn by the user. The clip may be configured to attach the device to sunglasses worn by the user.

These as well as other aspects and advantages will become apparent to those of ordinary skill in the art by reading the following detailed description, with reference where appropriate to the accompanying drawings. Further, it should be understood that the embodiments described in this summary and elsewhere are intended to be examples only and do not necessarily limit the scope of the present disclosure.

### BRIEF DESCRIPTION OF THE DRAWINGS

Various ones of the appended drawings merely illustrate example embodiments of the present disclosure and should not be considered as limiting its scope.

FIG. 1 is a perspective view of a user wearing a device according to an example embodiment.

FIG. 2 is a perspective view of a user wearing a device according to an example embodiment.

FIG. 3 is a perspective view of a user wearing a device according to an example embodiment.

FIG. 4 is a perspective view of a user wearing a device according to an example embodiment.

FIG. 5 is a schematic view of a device according to an example embodiment.

FIG. 6 is a schematic view of a device according to an example embodiment.

### DETAILED DESCRIPTION

Accordingly, embodiments of the present disclosure relate to a device for reducing face-touching. The device can help discourage people from touching their faces. After wearing the device for a certain period of time, people can be trained or conditioned to break the habit of frequently touching their faces. As such, it is possible to prevent the spread of serious respiratory diseases such as COVID-19.

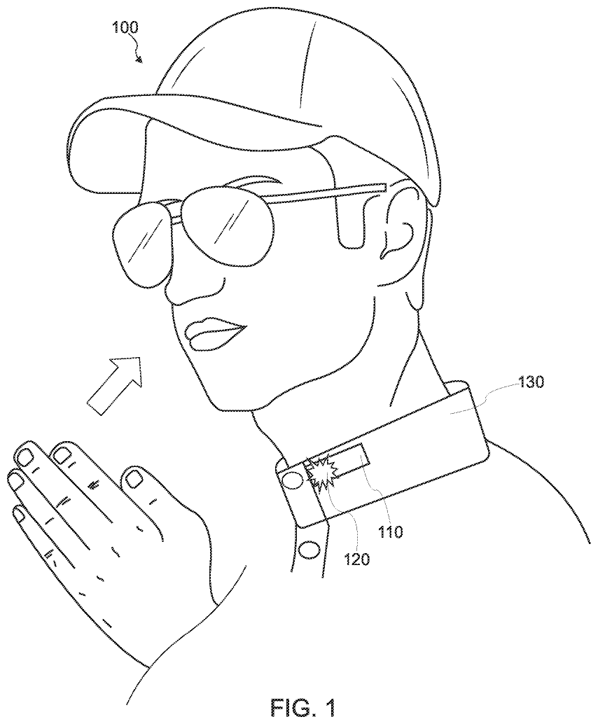

Turning to the figures, FIG. 1 shows a user 100 wearing a device 110 according to an example embodiment. The device 110 is attached to a collar 130 of a shirt worn by the user. When the user moves his hand toward his face, the device 110 notifies the user by generating a vibration output. In addition, the device can notify the user by emitting a light 120.

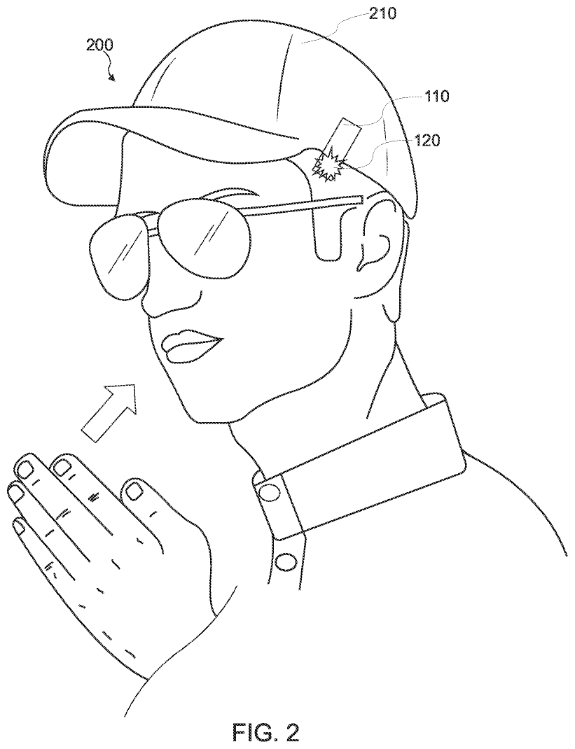

FIG. 2 shows a user 200 wearing the device 110. The device 110 is attached to a hat 210 worn by the user. When the user's hand is in close proximity to his face, the device 110 alerts the user by generating a vibration output. In addition, the device can alert the user by emitting a light 120.

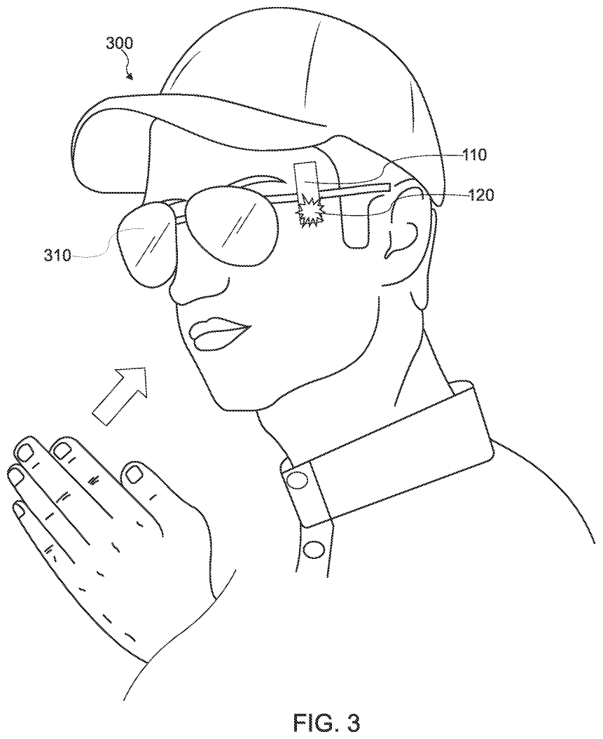

FIG. 3 shows a user 300 wearing the device 110. The device 110 is attached to sunglasses 310 worn by the user. When the user's hand is moved to be in close proximity to his face, the device notifies the user by generating a vibration output. In addition, the device can notify the user by emitting a light 120.

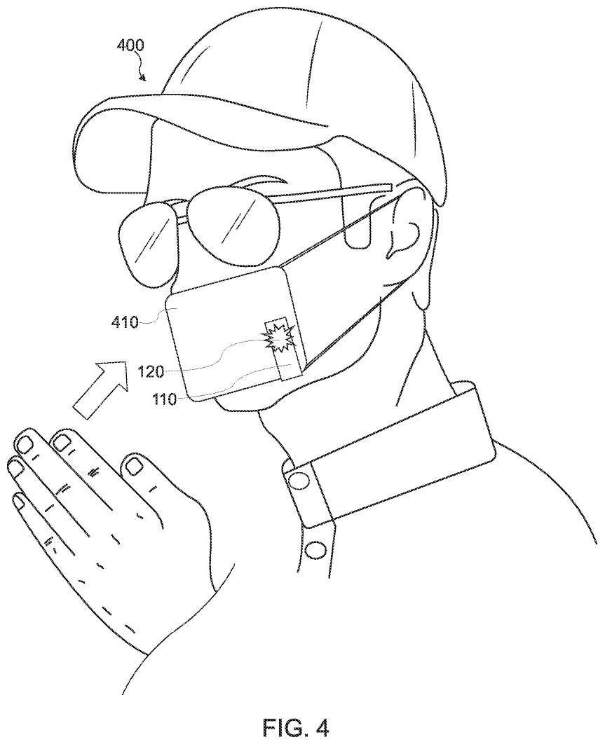

FIG. 4 shows a user 400 wearing the device 110. The device 110 is attached to a face mask 410 worn by the user. The face mask 410 may be a cloth mask or a disposable surgical mask, used to contain droplet spread from coughs, sneezes, and conversation. When the user's hand is moved to be in close proximity to his face, the device notifies the user by generating a vibration output. In addition, the device can notify the user by emitting a light 120.

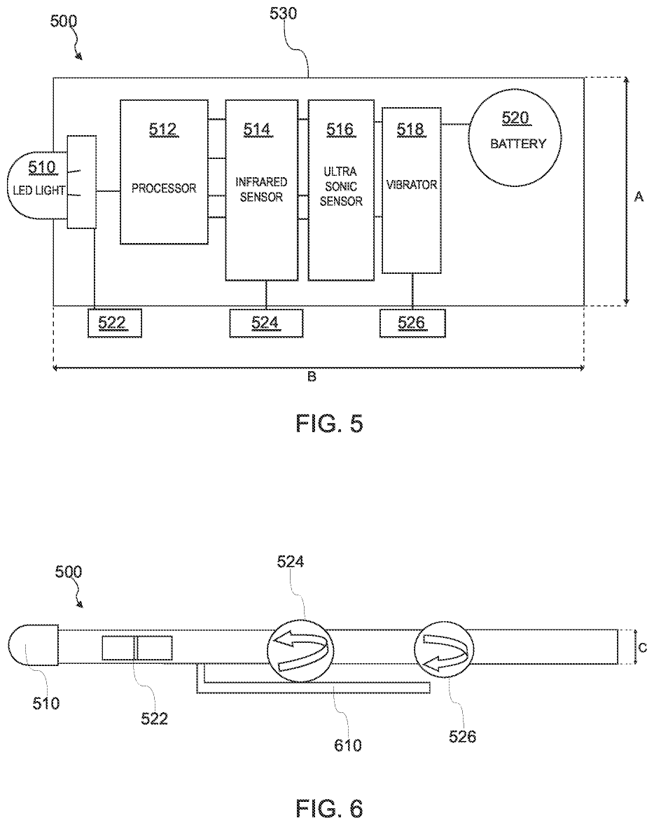

FIG. 5 shows the internal components of a device 500 according to an example embodiment of the disclosure. The device 500 includes an LED light 510, a processor 512, an infrared sensor 514, an ultrasonic sensor 516, a vibrator 518, and a battery 520. The processor 512, infrared sensor 514, ultrasonic sensor 516, vibrator 518, and battery 520 are each housed within a plastic case 530. As such, the LED light 510, processor 512, infrared sensor 514, ultrasonic sensor 516, vibrator 518, and battery 520 form an integrated unit. The device 500 is a stand-alone device that does not require the use of another device, such as a cell phone.

The LED light 510 extends somewhat from the surface of the plastic case 530 to make it more visible to the user when lit. Alternatively, the LED light may be arranged on a flexible wand so that the user can bend the wand and position the LED light in a desired location within the user's field of vision.

The infrared sensor 514 is a passive infrared sensor that measures infrared light radiating from objects in its field of view. The plastic case 530 has a small plastic window (not shown) through which infrared energy can enter.

The ultrasonic sensor 517 measures the distance to an object using ultrasonic sound waves. The ultrasonic sensor 517 includes a transducer to send an ultrasonic pulse and receive an echo of the pulse off an object. The ultrasonic sensor 517 receives sound waves through a small opening (not shown) in the case 530. The ultrasonic sensor 517 can complement the infrared sensor 514 since the ultrasonic sensor does not rely on visual components. For example, the ultrasonic sensor can “see” in the dark, through dust and dirt in varying light conditions. Either the ultrasonic sensor 517 or the infrared sensor 514 may be optionally omitted.

The processor 512 is coupled to a memory that instructs the processor 512 to perform certain functions. For instance, when the infrared sensor 514 and/or the ultrasonic sensor 517 detects the user's hand within a threshold proximity to the user's face, a signal is sent to the processor 512. After receiving these signals, the processor 512 sends a signal to the vibrator 518, which then generates a vibration output. In addition, the processor 512 can use an algorithm to increase the intensity of the vibration output as the user's hand gets closer to the user's face.

The processor 512 is electrically connected to each of the LED light 510, the infrared sensor 514, the ultrasonic sensor 516, the vibrator 518, and the battery 520.

The battery 520 can be a disposable button cell battery (e.g., a watch battery or coin battery). Alternatively, the battery 520 can be a rechargeable battery (e.g., a lithium-ion or lithium-polymer battery). When using a rechargeable battery, the device can include a charging interface, such as a USB-C port, for charging the battery.

The vibrator 518 is button-type, vibrating motor with a shaftless design. The vibration motor has a small size, for instance approximately 10 mm in diameter by 2.0 mm in height.

The device 500 further includes a light on/off switch 522 which activates and deactivates the LED light 510. When the switch 522 is in the “on” position, the LED light 510 is in an activated state. In this state, the processor 512 will send a signal to the LED light 510 causing it to light when the processor 512 receives a signal from the infrared sensor 514 and/or the ultrasonic sensor 517. When the switch 522 is in the “off” position, the LED light 510 is in a deactivated state, and will not light up even if the processor 512 receives a signal from one of the ultrasonic and infrared sensors.

The device 500 further includes a sensitivity control 524 (e.g., a twistable knob) for varying a detection range of the infrared sensor 514 and/or the ultrasonic sensor 517. Sensitivity can be set lower if the user is receiving false triggers, for example, from outside objects moving in the vicinity of the user. On the other hand, sensitivity can be set higher if the user is experiencing non-triggering issues, for example, if the device is not triggered when the user's hand is approaching the user's face. The user can adjust the threshold distance at which their hand triggers the device. For instance, the user can adjust the threshold proximity to somewhere between about 3 inches to 9 inches. The user may adjust the threshold proximity so that the device is triggered when the hand is near enough to the face that touching of the face was likely to occur.

The device 500 also includes a vibrator control 526 (e.g., a twistable knob) for controlling the intensity of the vibration output generated by the vibrator 518. This allows the user to vary the vibration strength depending on anatomy, device location, personal preference, etc.

Thus, the sensitivity control 524 and the vibrator control 526 allow the user to fine-tune (e.g., dial-in) the sensor sensitivity and vibration intensity, respectively.

In some embodiments, the device 500 may include a tiny speaker which emits a noise (e.g., a beep) when the user's hand is in close proximity to their face. The device 500 may further include an audio switch for activating and deactivating the speaker.

In other embodiments, the device 500 may include an on/off switch for powering the device. This on/off switch can be a slidable on/off switch.

FIG. 6 illustrates an edge view of the device 500. As shown there, the device 500 includes a clip 610 affixed to the plastic case 530. Thus, the clip 610 can form an integrated unit together with the other components of the device.

The clip 610 allows the device to be attached to an item worn by the user. The clip 610 may be a metal clip to provide good durability. The clip 610 can be used to attach the device 500 to a user somewhere in an appropriate region near the user's face, such that the device 500 can detect the user's hand within a threshold proximity (e.g., from about 3 to 6 inches) from the user's face. For instance, referring back to FIGS. 1 to 4, the device can be attached to a user's shirt collar, hat, sunglasses, face covering, or other item worn in the vicinity of the user's face. The window for the infrared sensor 514 and the opening for the ultrasonic sensor 517 can be pointed toward a region in which a user's hand approaches the user's face.

As mentioned earlier, FIG. 4 shows a device 110 attached to a mask 410. The device 110 is clipped to an edge of the mask 410. In an alternative embodiment, the mask can have a pocket sewn into the mask, so that the device 110 can be clipped to the pocket or inserted into the pocket.

Turning again to FIGS. 5 and 6, the device 500 has relatively small dimensions. For example, the device 500 may have a height A of about 2.5 cm, a length B of about 3.5 cm, and a width C of about 0.5 cm, where the term “about” means plus/minus 0.2 cm. Accordingly, it is made to easy to transport the device 500 and discretely attach it to various items worn by the user, such as sunglasses, a hat, collar, or face covering.

As shown in FIG. 6, the sensitivity control 524 and the vibration control 526 have different sizes. This can allow the user to more easily distinguish between the two controls when wearing the device.

While the disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions. For example, it is to be understood that the disclosure contemplates that, to the extent possible, one or more features of any embodiment can be combined with one or more features of any other embodiment.

1. A device for reducing face-touching, comprising:
a battery;
a sensor for detecting when a user's hand is within a threshold proximity to the user's face;
a vibrator configured to vibrate in response to the detection by the sensor that the user's hand is within the threshold proximity; and
a clip configured to attach the device to an item worn by the user,
wherein the battery, sensor, and vibrator are contained within the same casing.

2. The device according to claim 1, further comprising an LED light configured to light up when the sensor detects that the user's hand is within the threshold proximity.

3. The device according to claim 2, further comprising a switch for activating and deactivating the LED light.

4. The device according to claim 1, wherein the battery is a rechargeable battery.

5. The device according to claim 1, wherein the vibrator is configured to generate a vibration output with increasing intensity as the user's hand gets closer to the user's face.

6. The device according to claim 1, further comprising a switch for controlling the sensitivity of the sensor to change the threshold proximity.

7. The device according to claim 1, further comprising a switch for controlling a vibration intensity of the vibrator.

8. The device according to claim 1, further comprising a switch for activating and deactivating the LED light.

9. The device according to claim 1, wherein the sensor is an infrared sensor.

10. The device according to claim 1, wherein the sensor is an ultrasonic sensor.

11. The device according to claim 1, further comprising a processor configured to process information obtained from the sensor and to cause the vibrator to generate a vibration output.

12. The device according to claim 1, wherein the clip is configured to attach the device to a collar of a shirt worn by the user.

13. The device according to claim 1, wherein the clip is configured to attach the device to a hat worn by the user.

14. The device according to claim 1, wherein the clip is configured to attach the device to a mask worn by the user.

15. The device according to claim 1, wherein the clip is configured to attach the device to sunglasses worn by the user.
